(19) United States
(12) Patent Application Publication
(10) Pub. No.: US 2008/0202716 A1
(43) Pub. Date: Aug. 28, 2008

(54) Amphoteric 4-4'-Bis (Triazinylamino) Stilbene-2,2'-Disulfonic Acid Derivatives as Optical Brighteners for Paper

(76) Inventors: Goetz Scheffler, Grenzach-Wyhlen (DE); Rene Schlatter, Basel (CH)

Correspondence Address: JoAnn Villamizar;Ciba Corporation/Patent Department, 540 White Plains Road, P.O. Box 2005, Tarrytown, NY, 10591, US

(21) Appl. No.: 11/665,626

(22) PCT Filed: Oct. 10, 2005
(86) PCT No.: PCT/EP2005/055122

§ 371 Date: Apr. 17, 2007

(30) Foreign Application Priority Data
Oct. 20, 2004 (EP) 04105184.8

(51) Int. Cl. D21H 17/07; C07D 251/68

(52) U.S. Cl. 162/158; 544/193.2

(57) ABSTRACT

The present invention provides 4,4′-bis(triazinylamino)stilbene-2,2′-disulfonic acid derivatives of formula (1), compositions of amphoteric 4,4′-bis(triazinylamino)stilbene-2,2′-disulfonic acid derivatives, a process for their preparation, aqueous formulations thereof, their use as an optical brightener for paper and to paper treated with these derivatives.

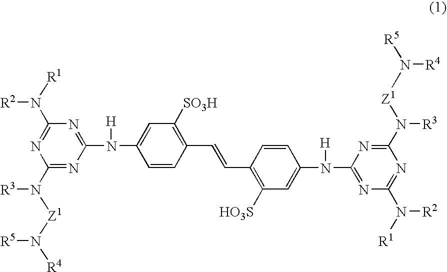

The present invention refers to amphoteric 4,4′-bis(triazinylamino)stilbene-2,2′-disulfonic acid derivatives and compositions of amphoteric 4,4′-bis(triazinylamino)stilbene-2,2′-disulfonic acid derivatives, a process for their preparation, aqueous formulations thereof, their use as an optical brightener for paper and to paper treated with these derivatives.

Paper treated with optical brighteners appears brighter and whiter, because the optical brighteners absorb ultraviolet light and reemit it as visible blue and violet light. Optical brighteners commonly used in paper making are 4,4′-bis(triazinylamino)stilbene-2,2′-disulfonic acid derivates.

WO 03/078406 and WO 04/046293, for example, describe amphoteric 4,4′-bis(triazinylamino)stilbene-2,2′-disulfonic acid derivates which show a whitening and brightening effect which is less diminished compared to anionic optical brightners in the presence of cationic polymers, which are used, for example, as retention aids, or other optical brighteners. The disadvantage of the amphoteric 4,4′-bis(triazinylamino)stilbene-2,2′-disulfonic acid derivates disclosed in WO 03/078406 and WO 04/046293 is their low water solubility. Thus, an aqueous formulation of these derivatives requires the presence of water-miscible organic solvents and/or other auxiliaries.

It is an object of the present invention to provide novel amphoteric optical brighteners, which show high water solubility, especially at or below pH below 11, and thus can be formulated as an aqueous solution in the absence of water-miscible organic solvents or other auxiliaries.

This object is achieved by the amphoteric 4,4′-bis(triazinylamino)stilbene-2,2′-disulfonic acid derivatives according to claims 1 and 9, by the compositions of amphoteric 4,4′-bis(triazinylamino)stilbene-2,2′-disulfonic acid derivatives according to claim 5, by the processes for their preparation according to claims 10 and 11, by the intermediates according to claims 12, 13 and 14, by the paper according to claim 16 and by the aqueous formulations according to claim 17.

The 4,4′-bis(triazinylamino)stilbene-2,2′-disulfonic acid derivatives of the present invention are compounds of formula

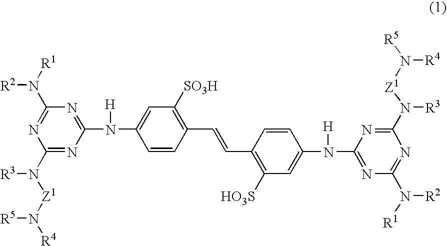

or salts thereof, in which
R¹ represents aryl, which is substituted with at least one hydroxy group, one carboxy group and/or one carboxy-C₁₋₄-alkyl (HOOC—C₁₋₄-alkyl) group, or R¹ represents C₁₋₆-alkyl, aralkyl or C₅₋₆-cycloalkyl, which are substituted with at least one carboxy group, whereby aryl may additionally be substituted with C₁₋₄-alkyl and/or C₁₋₄-alkoxy, and C₁₋₆-alkyl, aralkyl and C₅₋₆-cycloalkyl may additionally be substituted with hydroxy and/or C₁₋₄-alkoxy, and

R² represents hydrogen, C₁₋₄-alkyl, C₅₋₆-cycloalkyl, aryl, aralkyl or R¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy,

or
R¹ and R² together with the nitrogen to which they are attached complete a piperidino-, pyrrolidinyl- or morpholino ring, which is substituted with at least one carboxy group, and
Z¹ represents C₂₋₆-alkylene or C₅₋₆-cycloalkylene, whereby C₂₋₆-alkylene may be substituted with hydroxy and/or C₁₋₄-alkoxy, and may be interrupted by one or two oxygen atoms, whereby the two oxygens are not linked to each other, and
R³, R⁴ and R⁵ each independently of each other represent hydrogen, C₁₋₄-alkyl, C₅₋₆-cycloalkyl or −Z²-NR⁶R⁷, or R⁴ and R⁵ together with the nitrogen to which they are attached complete a morpholino- piperidino- or pyrrolidinyl ring, and whereby C₁₋₄-alkyl may be substituted with hydroxy, C₁₋₄-alkoxy, carbamoyl and/or cyano; Z² represents C₂₋₆-alkylene or C₅₋₆-cycloalkylene, whereby C₂₋₆-alkylene may be substituted with hydroxy and/or C₁₋₄-alkoxy, or may be interrupted by one or two oxygen atoms; R⁶ and R⁷ each independently of each other represent hydrogen, C₁₋₄-alkyl or C₅₋₆-cycloalkyl,
or
Z¹, R³ and R⁴ together with the nitrogens to which they are attached complete a piperazinyl ring,
with the proviso that when R¹ represents 4-carboxyphenyl and R² represents hydrogen, Z¹ is not ethylene, R⁴ and R⁵ are not methyl and R³ is not hydrogen; or Z¹ is not trimethylene, R⁴ and R⁵ do not together with the nitrogen to which they are attached complete a morpholino ring and R³ is not hydrogen.

Salts thereof can be salts formed by reacting the 4,4′-bis(triazinylamino)stilbene-2,2′-disulfonic acid derivative (1) with an alkaline metal, alkaline earth metal, ammonium or mono-, di-, tri- or tetraC₁₋₄-alkylammonium salt of a weak acid such as water or carbonic acid. Examples of alkaline metals are sodium, potassium and lithium. Examples of alkaline earth metals are magnesium, calcium and barium. Examples of mono-, di-, tri- or tetraC₁₋₄-alkylammonium are tetrabutylammonium and triethylammonium.

Aryl can be phenyl, pyridyl, pyrazinyl, pyrimidinyl, naphthyl, isoquinolyl, quinoxalinyl, quinazolinyl, phthalazinyl, quinolyl, pyrrolyl, furyl or thienyl. A carboxy-C₁₋₄-alkyl group can be carboxymethyl, 2-carboxyethyl, 3-carboxypropyl or 4-carboxybutyl. C₁₋₆-Alkyl can be methyl, ethyl, propyl, isopropyl, butyl, isobutyl, sec-butyl, tert-butyl, pentyl, isopentyl, 2-pentyl, 3-pentyl, 2-methylbutyl, 2,2-dimethylpropyl, 3-methyl-2-butyl, 2-methyl-2-butyl, hexyl, isohexyl, 2-hexyl, 3-hexyl, 3-methylpentyl, 2-methylpentyl, 3,3-dimethylbutyl, 2,3-dimethylbutyl, 2,2-dimethyl butyl, 2-ethyl butyl, 4-methyl-2-pentyl, 3-methyl-2-pentyl, 2-methyl-2-pentyl, 3,3-dimethyl-2-butyl, 2,3-dimethyl-2-butyl, 2-ethyl-2-butyl or 4-methyl-3-pentyl. Aralkyl can be benzyl or 2-phenylethyl. C₅₋₆-Cycloalkyl is cyclopentyl or cyclohexyl. C₁₋₄-Alkyl can be methyl, ethyl, propyl, isopropyl, butyl, isobutyl, sec-butyl or tert-butyl. C₁₋₄-Alkoxy can be methoxy, ethoxy, propoxy, isopropoxy, butoxy, isobutoxy, sec-butoxy or tert-butoxy. C₂₋₆-Alkylene can be ethylene, trimethylene, propylene, tetramethylene, ethylethylene, pentamethylene or hexamethylene. C₅₋₆-Cycloalkylene is cyclopentylene or cyclohexylene.

Preferably, R¹ represents phenyl which is substituted with at least one hydroxy group, one carboxy group and/or one carboxy-C₁₋₄-alkyl (HOOC—C₁₋₄-alkyl) group, or R¹ represents C₁₋₆-alkyl which is substituted with at least one carboxy group, whereby phenyl may additionally be substituted with C₁₋₄-alkyl and/or C₁₋₄-alkoxy, and C₁₋₆-alkyl may additionally be substituted with hydroxy and/or C₁₋₄-alkoxy, and

R² represents hydrogen, C₁₋₄-alkyl, or R¹, whereby C₁₋₄-alkyl may be substituted with hydroxy, C₁₋₄-alkoxy, carbamoyl and/or cyano, and
Z¹ represents C₂₋₆-alkylene, which may be substituted with hydroxy and/or C₁₋₄-alkoxy, and may be interrupted by one or two oxygen atoms, whereby the two oxygens are not linked to each other, and
R³ represents hydrogen or C₁₋₄-alkyl, whereby C₁₋₄-alkyl may be substituted with hydroxy, C₁₋₄-alkoxy, carbamoyl and/or cyano, and
R⁴ and R⁵ each independently of each other represent hydrogen, C₁₋₄-alkyl or −Z²-NR⁶R⁷, or together with the nitrogen to which they are attached complete a morpholino-, piperidino- or pyrrolidinyl ring, and whereby C₁₋₄-alkyl may be substituted with hydroxy and/or C₁₋₄-alkoxy; Z² represents C₂₋₆-alkylene, which may be substituted with hydroxy and/or C₁₋₄-alkoxy, or may be interrupted by one or two oxygen atoms; R⁶ and R⁷ each independently of each other represent hydrogen or C₁₋₄-alkyl,
or Z¹, R³ and R⁴ together with the nitrogens to which they are attached complete a piperazinyl ring.

More preferably, R¹ represents phenyl which is substituted with at least one hydroxy group and/or one carboxy group, or R¹ represents C₁₋₆-alkyl, which are substituted with at least one carboxy group, whereby phenyl may additionally be substituted with C₁₋₄-alkyl, C₁₋₄-alkoxy and/or C₁₋₄-alkylsulfonyl, and C₁₋₆-alkyl may additionally be substituted with hydroxy and/or C₁₋₄-alkoxy, and

R² represents hydrogen, C₁₋₄-alkyl or R¹, and
Z¹ represents C₂₋₄-alkylene, whereby C₂₋₄-alkylene may be substituted with hydroxy and/or C₁₋₄-alkoxy, and
R³ represents hydrogen or C₁₋₄-alkyl, whereby C₁₋₄-alkyl may be substituted with hydroxy and/or C₁₋₄-alkoxy, and
R⁴ and R⁵ each independently of each other represent hydrogen, C₁₋₄-alkyl or -Z²-NR⁶R⁷, whereby C₁₋₄-alkyl may be substituted with hydroxy and/or C₁₋₄-alkoxy; or R⁴ and R⁵ together with the nitrogen to which they are attached complete a morpholino ring; Z² represents C₂₋₄-alkylene, which may be substituted with hydroxy and/or C₁₋₄-alkoxy; R⁶ and R⁷ each independently of each other represent hydrogen or C₁₋₄-alkyl,
or Z¹, R³ and R⁴ together with the nitrogens to which they are attached complete a piperazinyl ring.

C₂₋₄-Alkylene can be ethylene, trimethylene, propylene, tetramethylene or ethylethylene.

Most preferably, R¹ represents phenyl or C₁₋₆-alkyl, which are substituted with one hydroxy or carboxy group; and R² represents hydrogen, methyl or R¹; and Z¹ represents C₂₋₃-alkylene; and R³ represents hydrogen, and

R⁴ and R⁵ each independently of each other represent hydrogen or C₁₋₄-alkyl, whereby C₁₋₄-alkyl may be substituted with hydroxy; or R⁴ and R⁵ together with the nitrogen to which they are attached complete a morpholino ring; or Z¹, R³ and R⁴ together with the nitrogens to which they are attached complete a piperazinyl ring.

C₂₋₃-alkylene is ethylene, trimethylene or propylene.

The composition of the present invention comprises 4,4′-bis(triazinylamino)stilbene-2,2′-disulfonic acid derivatives of the formulae

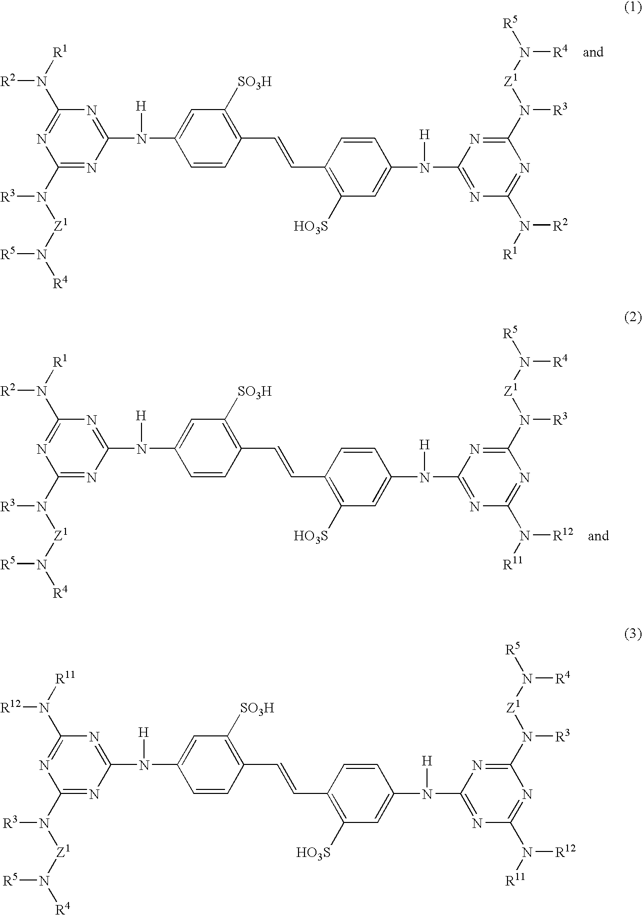

or salts thereof, in which R¹, R², Z¹, R³, R⁴ and R⁵ are as defined above, and
R¹¹ represents aryl, C₁₋₆-alkyl, aralkyl or C₅₋₆-cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆-alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆-cycloalkyl, aryl, aralkyl or R¹¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy.

Preferably, R¹¹ represents phenyl or C₁₋₆-alkyl, whereby phenyl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆-alkyl may be substituted with a hydroxy, a carboxy group, C₁₋₄-alkoxy, carbamoyl and/or cyano; and R¹² represents hydrogen, C₁₋₄-alkyl or R¹¹, whereby C₁₋₄-alkyl may be substituted with hydroxy, C₁₋₄-alkoxy, carbamoyl and/or cyano.

More preferably, R¹¹ represents phenyl, whereby phenyl may be substituted with a hydroxy group, a carboxy group, a carboxy-C₁₋₄-alkyl (HOOC—C₁₋₄-alkyl) group, C₁₋₄-alkyl and/or C₁₋₄-alkoxy; and R¹² represents hydrogen or C₁₋₄-alkyl.

Most preferably, R¹¹ represents phenyl, which is unsubstituted or substituted with one carboxy group, and R¹² represents hydrogen.

The 4,4′-bis(triazinylamino)stilbene-2,2′-disulfonic acid derivatives 1, 2 and 3 can be present in any molar ratio. Preferably, the amount of 4,4′-bis(triazinylamino)stilbene-2,2′-disulfonic acid derivative 1 is from 2 to 25% by mole based on the molar sum of 1, 2 and 3.

Also part of the invention is the 4,4′-bis(triazinylamino)stilbene-2,2′-disulfonic acid derivative of the formula

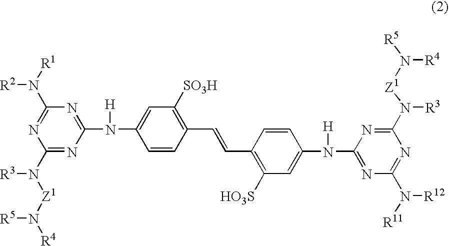

or salts thereof, in which R¹, R², Z¹, R³, R⁴, R⁵, R¹¹ and R¹² are as defined above.

Another part of the invention is a process for the preparation of the 4,4′-bis(triazinylamino)stilbene-2,2′-disulfonic acid derivatives (1) or salts thereof, which comprises the steps of
i) reacting a 4,4′-bis[(4,6-dihalotriazinyl)amino]stilbene-2,2′-disulfonic acid derivative of the formula

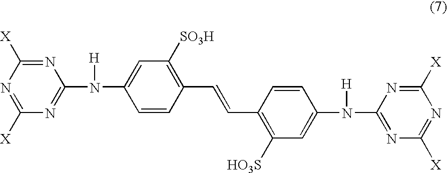

or salts thereof, in which X represents bromine, chlorine, fluorine or iodine,
with an amine of the formula HNR¹R² (8), in which R¹ and R² are as defined above, to yield a 4,4′-bis(triazinylamino)stilbene-2,2′-disulfonic acid derivative of the formula

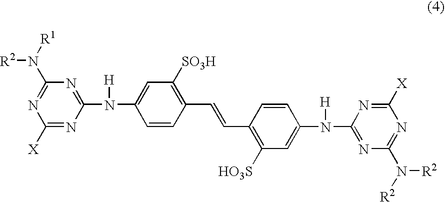

or salts thereof, in which R¹ and R² are as defined above,
ii) reacting the 4,4′-bis(triazinylamino)stilbene-2,2′-disulfonic acid derivative of the formula 4 obtained in step i) with an amine of the formula HNR³(Z¹-NR⁴R⁵) (9), in which R³, Z¹, R⁴ and R⁵ are as defined above, to yield the 4,4′-bis(tnazinylamino)stilbene-2,2′-disulfonic acid derivative of the formula 1 or salts thereof.

In step i) NHR¹R² (8) is usually added to a solution of the 4,4′-bis[(4,6-dihalotriazinylamino)]stilbene-2,2′-disulfonic acid derivate 7 in a mixture of an aqueous solution and a water-miscible organic solvent. An aqueous solution can be water or buffer. Examples of water-miscible organic solvents are acetone, methanol, ethanol, propanol, methyl ethyl ketone, dimethylsulfoxide, dimethylacetamide and dimethylformamide. In general, the molar ratio of 8/7 is from 2.0:1 to 2.2:1. Usually, the reaction is performed at a pH between 6.5 and 8.0, preferably between pH 7.0 and 7.5, and at a temperature between 60 and 80° C., preferably between 65 and 75° C. Preferably, the reaction is performed in a mixture of water and a water-miscible organic solvent and the pH is adjusted during the reaction by addition of a suitable base such as sodium hydroxide or potassium hydroxide. Preferably, methyl ethyl ketone is used as the water-miscible organic solvent and is removed by distillation after the reaction. Preferably X represents bromine or chlorine. More preferably, it represents chlorine. Preferably, the 4,4′-bis(triazinylamino)]stilbene-2,2′-disulfonic acid derivate 4 is precipitated from the reaction mixture and isolated by filtration.

Examples of most preferred HNR¹R² (8) are 2-, 3- and 4-aminophenol, anthranilic acid, 3- and 4-aminobenzoic acid, glycine, sarcosine, β-alanine, alanine, 4-aminobutyric acid, 2-aminobutyric acid, 2-aminoisobutyric acid, 5-aminovaleric acid, norvoline, valine, 6-aminocaproic acid, isoleucine, leucine, tert-leucine, norleucine and iminodiacetic acid.

In step ii) the amine HNR³(Z¹-NR⁴R⁵) (9) is usually added to an aqueous solution of the 4,4′-bis(triazinylamino)]stilbene-2,2′-disulfonic acid derivate 4. The aqueous solution can be water or buffer. In general, the molar ratio of 9/4 is from 2.0:1 to 3.0/1. The reaction is usually performed at a pH between 6.5 and 10, preferably at a pH between pH 7.0 and 9.0, and at a temperature between 70 and 100° C., preferably between 90 and 100° C. The pH is adjusted during the reaction by addition of a suitable base such as sodium hydroxide or potassium hydroxide. The 4,4′-bis(triazinylamino)stilbene-2,2′-disulfonic acid derivative 1 can be precipitated by acidifying the aqueous solution, preferably by acidifying it to a pH between 4.0 and 5.5. Optionally, the 4,4′-bis(triazinylamino)stilbene-2,2′-disulfonic acid derivative 1 can be recrystallized by dissolving it in water at a pH between 6.5 and 10.5, preferably between 7.5 and 9.5, and at a temperature between 50 and 100° C., preferably between 65 and 85° C., and precipitating it by acidifying the aqueous solution, preferably by acidifying it to a pH between 4.0 and 5.5.

Examples of more preferred amines NHR³(Z¹-NR⁴R⁵) (9) are 1,2-diaminopropane, 1-(2-aminoethyl)piperazine, diethylenetriamine, 1-(2-hydroxyethyl)piperazine, 3-(diethylamino)propylamine, 3,3′-diamino-N-methyldipropylamine, ethylenediamine, 3-(dimethylamino)propylamine, 4-(3-aminopropyl)morpholine, 1,3-diaminopropane, N-methylethylenediamine, 1,4-diaminobutane, N-ethylethylenediamine, N-methyl-1,3-propanediamine and N-(2-hydroxyethyl)ethylenediamine.

Examples of most preferred amines NHR³(Z¹-NR⁴R⁵) (9) are 1-(2-hydroxyethyl)piperazine, 3-(diethylamino)propylamine, 3,3′-diamino-N-methyldipropylamine, ethylenediamine, 3-(dimethylamino)propylamine, 4-(3-aminopropyl)morpholine, 1,3-diaminopropane, N-methylethylenediamine, 1,4-diaminobutane, N-ethylethylenediamine and N-methyl-1,3-propanediamine

The 4,4′-bis[(4,6-dihalotriazinyl)amino]stilbene-2,2′-disulfonic acid derivative of the formula 7 can be obtained by reacting 4,4′-diaminostilbene-2,2′-disulfonic acid with cyanuric halide in a mixture of an aqueous solution and a water-miscible organic solvent. An aqueous solution and a water-miscible organic solvent are defined above. Preferably, 4,4′-bis[(4,6-dichlorotriazinyl)amino]stilbene-2,2′-disulfonic acid derivative of the formula 7 is prepared by reacting 4,4′-diaminostilbene-2,2′-disulfonic acid with cyanuric chloride. The reaction can be carried out at a pH between 3.5 and 6.5, preferably between 4.5 and 5.5, and at a temperature between 5 and 15° C., preferably between 5 and 10° C. The 4,4′-bis[(4,6-dihalotriazinyl)amino]stilbene-2,2′-disulfonic acid derivative 7 is either isolated from the reaction mixture before used step i) or prepared in situ and used in step i) without prior isolation.

Alternatively, the 4,4′-bis(triazinylamino)stilbene-2,2′-disulfonic acid derivatives 1 or salts thereof can also be prepared by reacting the 4,4′-bis[(4,6-dihalotriazinyl)amino]stilbene-2,2′-disulfonic acid derivative 4 or salts thereof in a first step with HNR³(Z¹-NR⁴R⁵)(9) and in a second step with HNR¹R² (8).

Also part of the invention is a process for the preparation of the composition comprising 4,4′-bis(triazinylamino)stilbene-2,2′-disulfonic acid derivatives 1, 2 and 3 of the present invention, or salts thereof, which comprises the steps of
i) reacting a 4,4′-bis[(4,6-dihalotriazinylamino)]stilbene-2,2′-disulfonic acid derivate of the formula

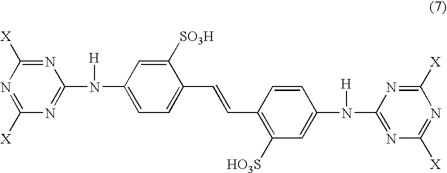

or salts thereof, in which X represents bromine, chlorine, fluorine or iodine,
with a mixture of amines of the formulae HNR¹R² (8), and HNR¹¹R¹² (10), in which R¹, R², R¹¹ and R¹² are as defined above, to yield a composition comprising 4,4′-bis(triazinylamino)stilbene-2,2′-disulfonic acid derivatives of the formulae

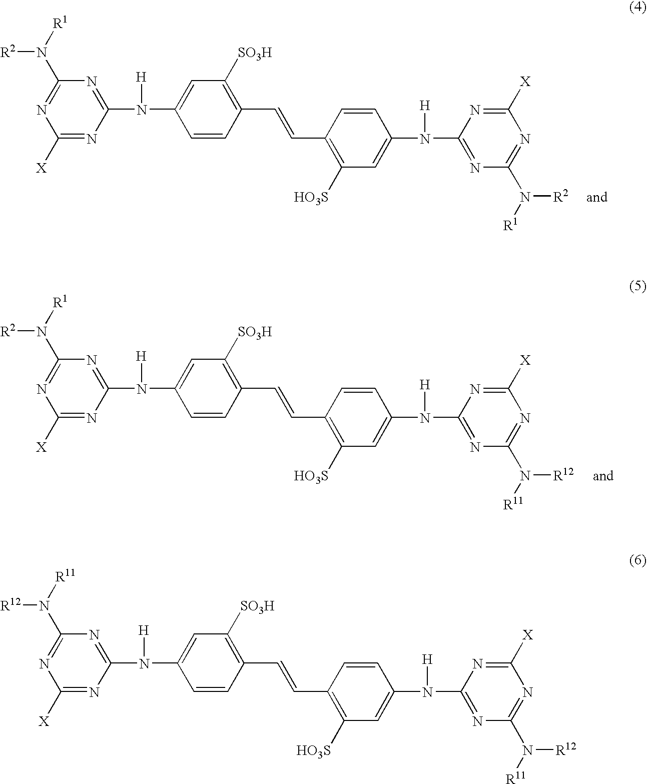

or salts thereof, in which R¹, R², R¹¹, R¹² and X are as defined above
ii) reacting the composition comprising 4,4′-bis(triazinylamino)stilbene-2,2′-disulfonic acid derivatives of the formulae 4, 5 and 6 or salts thereof obtained in step i) with an amine of the formula HNR³(Z¹-NR⁴R⁵) (9), in which R³, Z¹, R⁴ and R⁵ are as defined above, to yield the composition of 4,4′-bis(triazinylamino)stilbene-2,2′-disulfonic acid derivatives of the formulae 1, 2 and 3.

In general, step i) and ii) are performed as described above. In step i) the amines of the formulae HNR¹R² (8) and HNR¹¹R¹² (10) are usually added simultaneously to the solution of the 4,4′-bis[(4,6-dihalotriazinylamino)]stilbene-2,2′-disulfonic acid derivate 7 in a mixture of an aqueous solution and a water-miscible organic solvent. Preferably, the molar ratio of 10/8 is from 1:1 to 5:1, and the molar ratio of (8+10)/7 is from 2.0:1 to 2.2:1. Preferably, X represents chlorine or bromine. More preferably, it represents chlorine.

Alternatively, the composition comprising 4,4′-bis(triazinylamino)stilbene-2,2′-disulfonic acid derivatives 1, 2 and 3 or salts thereof can be prepared by reacting the 4,4′-bis[(4,6-dihalotriazinylamino)]stilbene-2,2′-disulfonic acid derivate 7 or salts thereof in a first step with HNR³(Z¹-NR⁴R⁵) (9) and in a second step with the mixture of HNR¹R² (8) and HNR¹¹R¹² (10).

Also part of the invention are the intermediate 4,4′-bis(triazinylamino)stilbene-2,2′-disulfonic acid derivatives of the following formulae

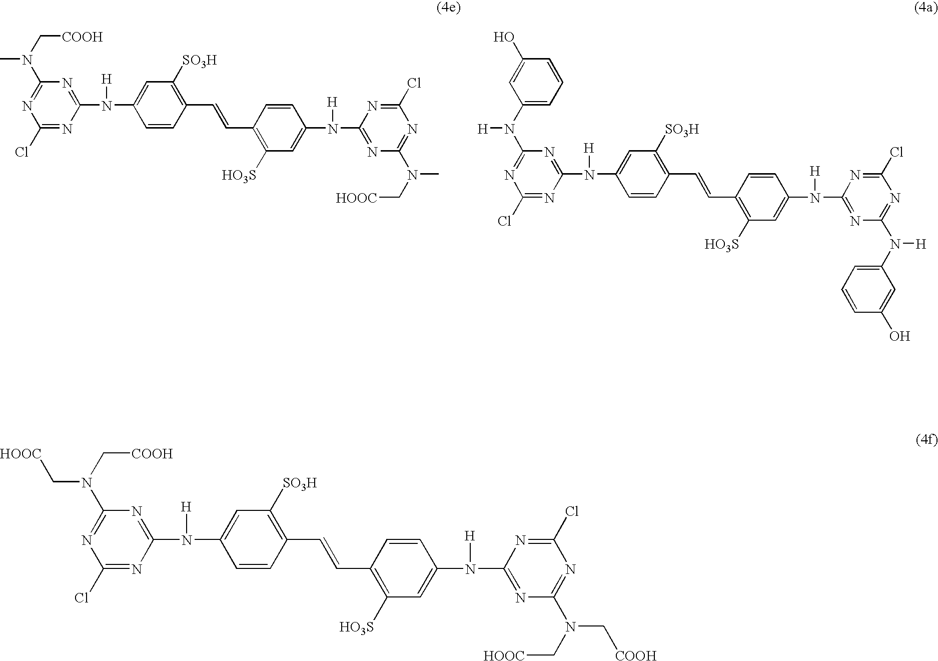

or salts thereof.

Another part of the invention are the intermediate 4,4′-bis(triazinylamino)stilbene-2,2′-disulfonic acid derivatives of the formula 5 or salts thereof and the compositions of intermediate 4,4′-bis(triazinylamino)stilbene-2,2′-disulfonic acid derivatives of the formulae 4, 5 and 6 or salts thereof. In intermediate 4,4′-bis(triazinylamino)stilbene-2,2′-disulfonic acid derivatives of the formula 5 and in compositions of intermediate 4,4′-bis(triazinylamino)stilbene-2,2′-disulfonic acid derivatives of the formulae 4, 5 and 6, R¹, R², R¹⁰ and R¹¹ are as indicated above.

Another part of the present invention is the use of the 4,4′-bis(triazinylamino)stilbene-2,2′-disulfonic acid derivatives of the formulae 1 or 2 or salts thereof, or of the composition comprising the 4,4′-bis(triazinylamino)stilbene-2,2′-disulfonic acid derivatives of the formulae 1, 2 and 3 or salts thereof as an optical brightener for paper.

Also part of the invention is paper treated with the 4,4′-bis(triazinylamino)stilbene-2,2′-disulfonic acid derivatives of the formulae 1 or 2 or salts thereof, or with the composition comprising the 4,4′-bis(triazinylamino)stilbene-2,2′-disulfonic acid derivatives of the formulae 1, 2 and 3 or salts thereof. Paper treated with a 4,4′-bis(triazinylamino)stilbene-2,2′-disulfonic acid derivative 1 or 2 or with a composition comprising 4,4′-bis(triazinylamino)stilbene-2,2′-disulfonic acid derivatives 1, 2 and 3 can be prepared by applying a formulation of 4,4′-bis(triazinylamino)stilbene-2,2′-disulfonic acid derivative 1 or a formulation of a composition comprising 4,4′-bis(triazinylamino)stilbene-2,2′-disulfonic acid derivatives 1, 2 and 3 to the wet end part of the paper-making machine. The formulation can be a solid or an aqueous formulation. Preferably, it is an aqueous formulation.

The aqueous formulation is also part of the invention. This formulation can comprise the 4′-bis(triazinylamino)stilbene-2,2′-disulfonic acid derivative 1 or the composition comprising 4,4′-bis(triazinylamino)stilbene-2,2′-disulfonic acid derivatives 1, 2 and 3, water or buffer and optionally a water-miscible organic solvent and/or auxiliaries. Examples of water-miscible organic solvents are acetone, methanol, ethanol, propanol, methyl ethyl ketone, dimethylsulfoxide, dimethylacetamide and dimethylformamide. Examples of auxiliaries are urea, ethanolamine or polyglycol. Preferably the formulations are solutions of the 4,4′-bis(triazinylamino)stilbene-2,2′-disulfonic acid derivative 1 or of the composition comprising 4,4′-bis(triazinylamino)stilbene-2,2′-disulfonic acid derivatives 1, 2 and 3 in water or buffer in the absence of water-miscible organic solvents. More preferred are formulations of the 4,4′-bis(triazinylamino)stilbene-2,2′-disulfonic acid derivative 1 or of the composition comprising 4,4′-bis(triazinylamino)-stilbene-2,2′-disulfonic acid derivatives 1, 2 and 3 in water having a pH between 8.5 and 11.0.

The amphoteric 4,4′-bis(triazinylamino)stilbene-2,2′-disulfonic acid derivatives of formulae 1 or 2 or the composition comprising 4,4′-bis(triazinylamino)stilbene-2,2′-disulfonic acid derivatives 1, 2 and 3 show a high water solubility, especially at or below pH 11, and thus can be formulated as an aqueous solution. These optical brighteners also show a good substantivity towards fibres and their whitening and brightening effect is less diminished compared to anionic optical brightners by the presence of cationic polymers or other cationic optical brightners.

### EXAMPLES

### Examples 1 to 6

Preparation of

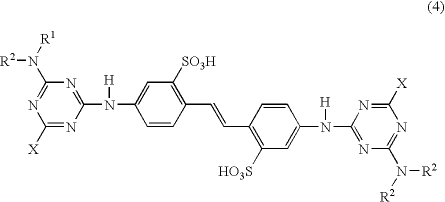

in which the sulfonic acid groups are in form of the sodium salt,
X is chlorine,
and

TABLE 1

| Example No | *—NR¹R² | Compound |
|---|---|---|
| 1 | 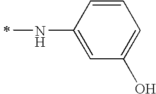 | 4a |
| 2 | 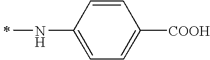 | 4b |
| 3 | 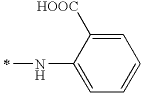 | 4c |
| 4 | 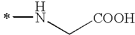 | 4d |
| 5 | 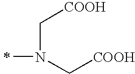 | 4e |
| 6 | 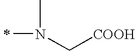 | 4f |

### Procedure for 4a to 4d

A solution of 4,4′-diamino-2,2′-stilbenedisulfonic acid (119 g, 0.32 mol) in aqueous soda (945 g) is added to a solution of cyanuric chloride (120 g, 0.65 mol) in water (400 g) and methyl ethyl ketone (753 g) at pH 4.5 to 5.5 (adjusted by addition of 20% (w/v) aqueous soda) and at 5 to 10° C. within 70 minutes. The mixture is stirred for further 10 minutes. NHR¹R² (8) (0.65 mol) is added at pH 7.0 to 7.5 (adjusted by addition of 50% (w/v) aqueous NaOH) and at 10 to 20° C. The mixture is warmed to 70° C. at pH 7.0 to 7.5 within 60 minutes, and stirred for further 10 minutes. Methyl ethyl ketone is removed by distillation. The remaining mixture is cooled to room temperature and left at room temperature overnight whereupon a precipitate is obtained. The precipitate is suction filtered, washed with 5% (w/w) brine and dried at 70 to 80° C. to afford 4 as yellow crystals.

### Procedure for 4e:

A solution of 4,4′-diamino-2,2′-stilbenedisulfonic acid, disodium salt (135 g, 0.325 mol) in water (786.9 g) is added to a solution of cyanuric chloride (120 g, 0.65 mol) in water (400 g) and methyl ethyl ketone (753 g) at pH 4.5 to 5.5 (adjusted by addition of 20% (w/v) aqueous soda) and at 5 to 10° C. within 55 minutes. The mixture is stirred for further 10 minutes. NHR¹R² (8) (0.65 mol) is added at pH 7.0 to 7.5 (adjusted by addition of 50% (w/v) aqueous NaOH) and at 10 to 20° C. The mixture is warmed to 70° C. at pH 7.0 to 7.5 within 60 minutes, and stirred for further 10 minutes. Methyl ethyl ketone is removed by distillation. The remaining mixture is cooled to room temperature and left at room temperature overnight whereupon a precipitate is obtained. The precipitate is suction filtered, washed with 5% (w/w) brine and dried at 70 to 80° C. to afford 4e as yellow crystals.

### Procedure for 4f:

A solution of 4,4′-diamino-2,2′-stilbenedisulfonic acid (119 g, 0.32 mol) in aqueous soda (945 g) is added to a solution of cyanuric chloride (120 g, 0.65 mol) in water (400 g) and methyl ethyl ketone (753 g) at pH 4.5 to 5.5 (adjusted by addition of 20% (w/v) aqueous soda) and at 5 to 10° C. within 70 minutes. The mixture is stirred for further 10 minutes. NHR¹R² (8) (0.65 mol) is added at pH 7.0 to 7.5 (adjusted by addition of 50% (w/v) aqueous NaOH) and at 10 to 20° C. The mixture is warmed to 70° C. at pH 7.0 to 7.5 within 60 minutes, and stirred for further 10 minutes. Methyl ethyl ketone is removed by distillation. The remaining mixture (1710 g) is cooled to room temperature. No precipitation occurred.

### Examples 7 to 19

Preparation of

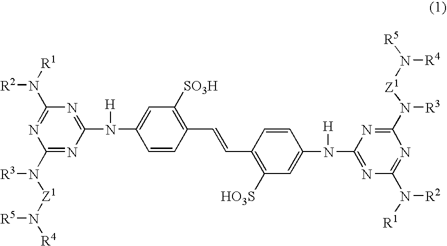

TABLE 2

| Example | Starting | | | Com- |
|---|---|---|---|---|
| No | material | *—NR¹R² | *—NR¹³(Z¹—NR⁴R⁵) | pound |
| 7 | 4a | 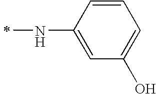 | 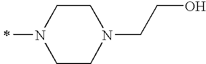 | 1a |
| 8 | 4a | 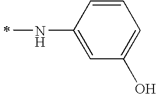 | 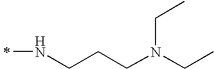 | 1b |
| 9 | 4b | 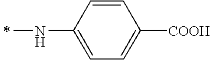 | 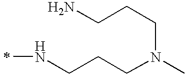 | 1c |
| 10^{a} | 4b | 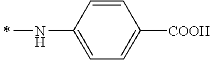 | 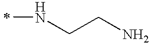 | 1d |
| 11 | 4b | 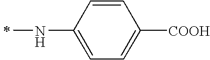 | 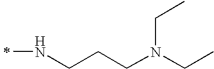 | 1e |
| 12 | 4b | 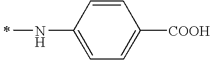 | 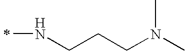 | 1f |
| 13^{b} | 4c | 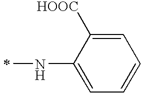 | 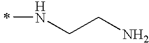 | 1g |
| 14 | 4c | 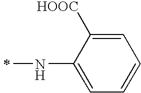 | 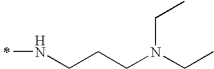 | 1h |
| 15 | 4c | 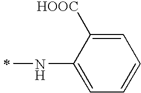 | 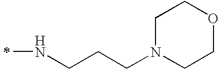 | 1i |
| 16 | 4c | 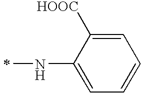 | 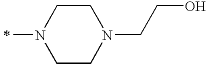 | 1j |
| 17 | 4d | 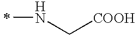 | 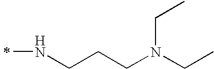 | 1k |
| 18 | 4d | 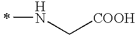 | 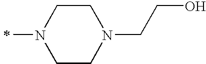 | 1l |
| 19 | 4f | 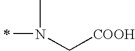 | 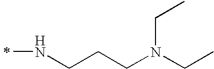 | 1m |

^{a}6.2 Mol equivalents of ethylenediamine were employed.
^{b}59 Mol equivalents of ethylenediamine were employed.

### Procedure for 1a to 1l:

NH₂R-Z′-NR⁴R⁵ (9) (2.4 to 3 mol equivalents with regard to 4) is added to a 13 to 17% (w/w) solution of 4 in water at pH 7.0 to 9.0 (adjusted by addition of 50% (w/v) aqueous NaOH) and at 70° C. The mixture is warmed to 97° C. at pH 8.0 to 10.0, and stirred under these conditions until 4 could not be detected anymore. The mixture was cooled to 70° C., and the pH is adjusted to 4.5 with concentrated HCl. The mixture is further cooled until a precipitate occurs. If a precipitate does not occur at temperatures above room temperature, the mixture is cooled to room temperature and left to stand overnight. The obtained precipitate is suction filtered and washed with 2.5% (w/w) brine. The filter cake is redissolved in water at pH 7.5 to 9.5 and 70 to 80° C. to yield an 8 to 16% (w/w) solution of the filter cake in water and reprecipitated by pH adjustment to 4.5 to 5.0. The precipitate is suction filtered, washed with 2.5% (w/w) brine and dried at 70 to 80° C. to yield 1 as yellow crystals.

### Procedure for 1 m:

NH₂R-Z′-NR⁴R⁵ (9) (2.4 mol equivalents with regard to 4f) is added to the reaction mixture containing 4f obtained as described in example 6 at pH 7.5 0 (adjusted by addition of 50% (w/v) aqueous NaOH) at 70° C. The mixture is warmed to 97° C. at pH 8.0 to 10.0, and stirred under these conditions until 4f could not be detected anymore. The mixture was cooled to 70° C., and the pH is adjusted to 5.5 with concentrated HCl. The mixture is further cooled until a precipitate occurs. The obtained precipitate is suction filtered and washed with 2.5% (w/w) brine to yield 1m as yellow crystals.

### Examples 20 to 24

Preparation of compositions comprising

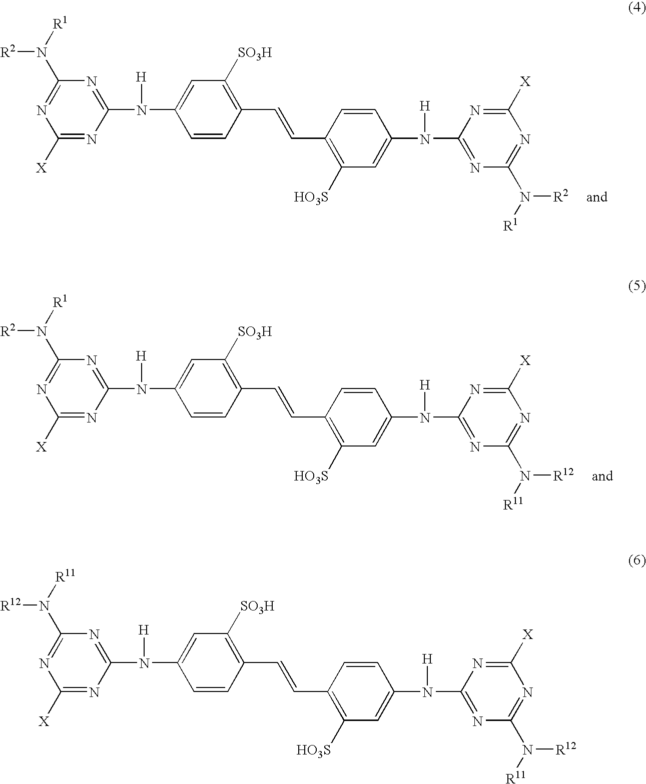

in which the sulfonic acid groups are in form of the sodium salt,
X is chlorine and

TABLE 3

| Ex- | | | |
|---|---|---|---|
| am- | | | |
| ple | | | |
| No | *—NR¹R² | *—NR¹¹R¹² | Compounds^{e} |
| 20 | 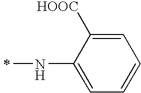 | 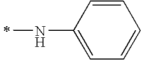 | 4c, 5a, 6a(23:42:31) |
| 21 | 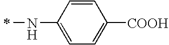 | 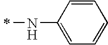 | 4b, 5b, 6a(33:34:25) |
| 22 | 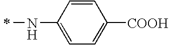 | 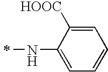 | 4b, 5c, 6b(27:46:22) |
| 23 | 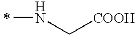 | 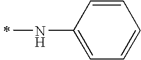 | 4d, 5d, 6a(18:16:53) |
| 24 | 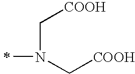 | 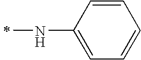 | 4e, 5e, 6a(29:24:32) |
| 25^{d} | 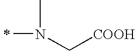 | 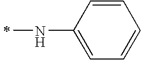 | 4f, 5f, 6a(2:23:70) |

^{c}6b is identical to 4c.
^{d}The molar ratio of sarcosine/aniline is 1:4 and the molar ratio of (sarcosine + aniline)/7 is 2.1:1.
^{e}[HPLC area %] at 350 nm.

### Procedure:

A solution of 4,4′-diamino-2,2′-stilbenedisulfonic acid (115 g, 0.31 mol) in aqueous soda (951 g) is added to a solution of cyanuric chloride (120 g, 0.65 mol) in water (400 g) and methyl ethyl ketone (753 g) at pH 4.5 to 5.5 and at 5 to 10° C. within 70 minutes. The mixture is stirred for further 10 minutes. NHR¹R² (8) (0.325 mol) and NHR¹¹R¹² (10) (0,325 mol) are added simultaneously at pH 7.0 to 7.5 and at 10 to 20° C. The mixture is warmed to 70° C. at pH 7.0 to 7.5 within 60 minutes, and stirred for further 10 minutes. Methyl ethyl ketone is removed by distillation. The remaining mixture is cooled to 45° C. and diluted with half the amount of brine. The solid is suction filtered, washed with 5% (w/w) brine and dried at 70 to 80° C. to afford a composition comprising 4, 5 and 6 as yellow crystals.

### Examples 26 to 31

Preparation of compositions comprising

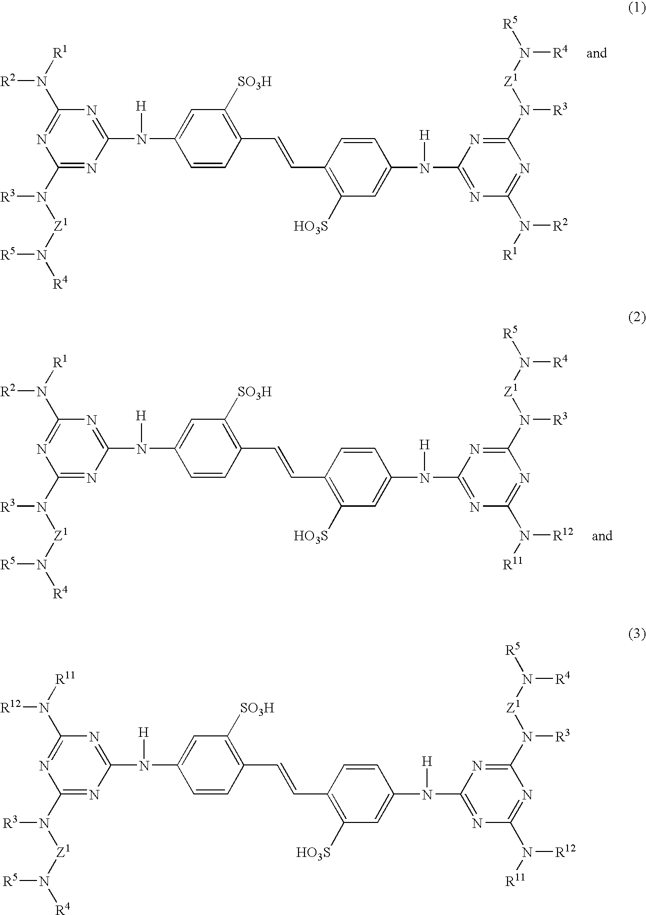

in which *—NR³(Z¹ NR⁴R⁵) is

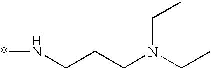

and

TABLE 4

| | Starting | | | |
|---|---|---|---|---|
| | material = | | | |
| | product | | | |
| Example | from | | | |
| No | example | *—NR¹R² | *—NR¹¹R¹² | Compounds^{e} |
| 26 | 17 | 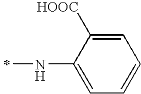 | 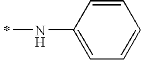 | 1h, 2a, 3a(18:39:35) |
| 27 | 18 | 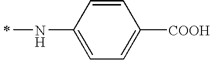 | 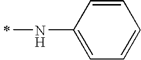 | 1e, 2b, 3a(33:33:25) |
| 28^{e} | 19 | 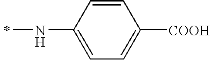 | 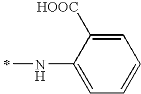 | 1e, 2c, 3b(24:45:23) |
| 29 | 20 | 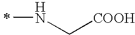 | 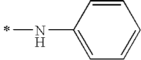 | 1k, 2d, 3a(16:15:53) |
| 30 | 21 | 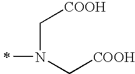 | 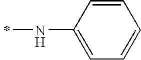 | 1m, 2e, 3a(22:26:32) |
| 31 | 22 | 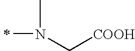 | 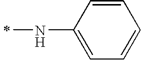 | 1n, 2f, 3a(2:22:65) |

^{e}[HPLC area %] at 350 nm.
^{f}6b is identical to 4c and 3b is identical to 1h.

### Procedure:

NH₂R³-Z¹-NR⁴R⁵ (9) (2.4 to 3 mol equivalents with regard to the mixture of 4, 5 and 6) is added to a 13 to 17% (w/w) solution of the mixture of 4, 5 and 6 in water at pH 7.0 to 9.0 and at 70° C. The mixture is warmed to 97° C. at pH 8.0 to 10.0, and stirred under these conditions until the mixture of 4, 5 and 6 can not be detected anymore. The mixture is cooled to 70° C., and the pH is adjusted to 4.5 with concentrated HCl. The mixture is further cooled until a precipitate occurs. If a precipitate does not occur at temperatures above room temperature, the mixture is cooled to room temperature and left to stand overnight. The obtained precipitate is suction filtered, washed with 2.5% (w/w) brine and dried at 70 to 80° C. to yield the mixture of 1, 2 and 3 as yellow crystals.

### Examples 32 to 38

### Measurement of Water-Solubility Behaviour of 4,4′-bis(triazinylamino)stilbene-2,2′-disulfonic acid derivatives 1

4,4′-Bis(triazinylamino)stilbene-2,2′-disulfonic acid derivatives 1 (0.5 g) are mixed with buffer (17 mM potassium dihydrogenphosphate, 43 mM di-sodium tetraborate, pH 9) (50 mL) and stirred for 2 hours at room temperature. After settlement of undissolved solids, 2 mL of the supernatant was filtered through a 0.2 μm filter and either used directly or diluted with water to a total volume of 1000 mL. The absorption of the supernatant or the diluted solution is measured at 350 nm using a UV-spectrometer.

TABLE 5

| Example | Com- | | | |
|---|---|---|---|---|
| No | pound | *—NR¹R² | *—NR³(Z¹—NR⁴R⁵) | absorption |
| 32 | 1a | 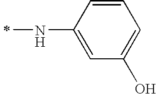 | 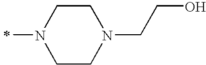 | 0.047^{g} |
| 33 | 1b | 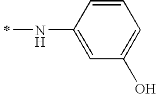 | 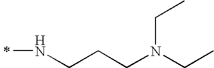 | 0.078^{h} |
| 34 | 1e | 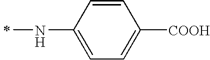 | 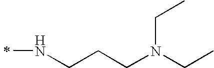 | 1.072^{g} |
| 35 | 1h | 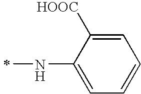 | 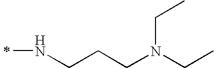 | 0.780^{g} |
| 36 | 1i | 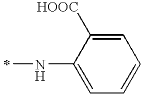 | 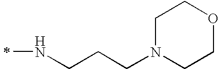 | 1.036^{g} |
| 37 | 1k | 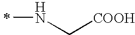 | 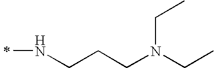 | 1.054^{g} |
| 38 | 1l | 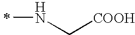 | 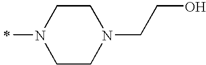 | 1.110^{g} |

^{g}Absorption of diluted solution measured.
^{h}Absorption of supernatant measured.

### Example 39

Measurement of the water-solubility behaviour of a mixture 1, 2 and 3 The same procedure as described for examples 32 to 38 is repeated, except that instead 0.5 g compound 1, 0.5 g of the mixture 1, 2 and 3 is used.

TABLE 6

| Example | Com- | *—NR¹R²/ | | |
|---|---|---|---|---|
| No | pounds | *—NR¹¹R¹² | *—NR³(Z¹—NR⁴R⁵) | absorption |
| 39 | 1e,2c,3b (24:45:23) | 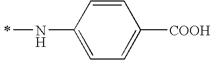 | 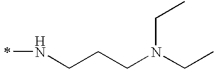 | 1.115^{g} |
| | | 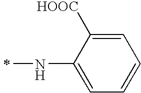 | | |

^{g}Absorption of diluted solution measured.

### Comparative Examples 1 to 6

Measurement of the water-solubility behaviour of compounds 11

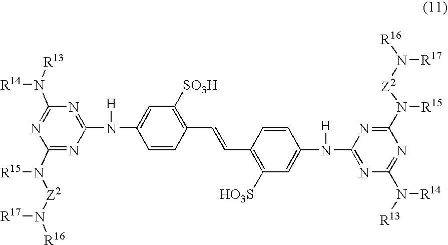

TABLE 7

| | | | | |
|---|---|---|---|---|
| Comp. | | | | |
| Example | Com- | | | |
| No | pound | *—NR¹³R¹⁴ | *—NR¹⁵(Z²—NR¹⁶R¹⁷) | absorption |

| | | | | |
|---|---|---|---|---|
| 1 | 11a | 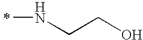 | 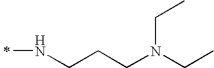 | 0.079^{g} |
| 2 | 11b | 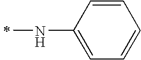 | 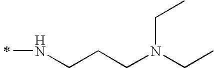 | 0.041^{h} |
| 3 | 11c | 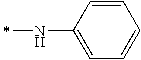 | 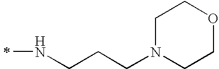 | 0.050^{g} |
| 4 | 11d | 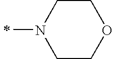 | 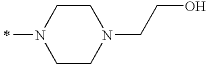 | 0.100^{g} |
| 5 | 11e | 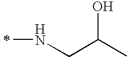 | 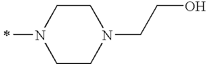 | 0.194^{g} |
| 6 | 11f | 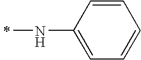 | 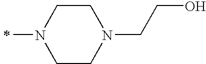 | 0.205^{h} |

^{g}Absorption of diluted solution measured.
^{h}Absorption of supernatant measured.

### Discussion

The higher the absorption, the higher is the concentration of 1, of the mixture of 1, 2 and 3, respectively of 11, in the solution, and the higher is the water-solubility of these compounds.

The absorption of a diluted solution of 1a (example 32) is 0.047. As the absorption is proportional to the concentration of the solution, the absorption of the supernatant of 1a should theoretically be 500×0.047=23.5. In contrast, the absorption of the supernatant of 11f (comparative example 6), which is the analogous compound without the hydroxyl group, is only 0.205.

The absorption of the supernatant of 1b (example 33) is 0.078, whereas the absorption of the supernatant of 11b (comparative example 2), which is the analogous compound without the hydroxyl group, is only 0.041.

The absorption of a diluted solution of 1e (example 34) is 1.072, and thus the absorption of the supernatant of 1e should theoretically be 500×1.072=536, whereas the absorption of the supernatant of 11b (comparative example 2), which is the analogous compound without the carboxy group, is only 0.041.

The absorption of a diluted solution of 1 h (example 35) is 0.780, and thus the absorption of the supernatant of 1e should theoretically be 500×0.780=390, whereas the absorption of the supernatant of 11b (comparative example 2), which is the analogous compound without the carboxy group, is only 0.041.

The absorption of a diluted solution of 1i (example 36) is 1.036, and thus the absorption of the supernatant of 1i should theoretically be 500×1.036=518, whereas the absorption of the diluted solution of 11c (comparative example 3), which is the analogous compound without the hydroxyl group, is only 0.050.

The absorption of a diluted solution of 1 k (example 37) is 1.054, whereas the absorption of the diluted solution of 1a (comparative example 1), which differs from 1k in that the carboxy group is replaced by a hydroxymethyl group, is only 0.079.

The absorption of a diluted solution of 1l (example 38) is 1.110, whereas the absorption of the diluted solution of 11d (comparative example 4), which differs from 1l in that the (carboxymethyl)amino group is replaced by a morpholino group, is only 0.100.

The absorption of a diluted solution of 11 (example 38) is 1.110, whereas the absorption of the diluted solution of 11e (comparative example 5), which differs from 11 in that the carboxy group is replaced by a 1-hydroxyethyl group, is only 0.194.

The absorption of a diluted solution of 1e, 2c and 3b (example 39) is 1.115, and thus the absorption of the supernatant of the mixture of 1e, 2c and 3b should theoretically be 500×1.115=575, whereas the absorption of the supernatant of 11b (comparative example 2), which differs from the mixture in that it does not have a carboxy group, is only 0.041.

Thus, it can be clearly seen that the 4,4′-bis(triazinylamino)stilbene-2,2′-disulfonic acid derivatives of the present invention show higher water-solubilities than comparable compounds, which are already known.

1. A 4,4′-bis(triazinylamino)stilbene-2,2′-disulfonic acid derivative of the formula 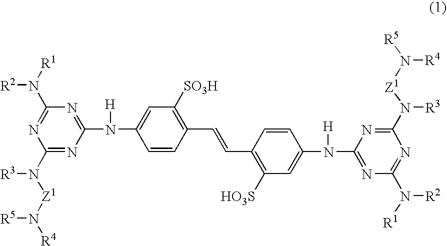
or salts thereof, in which
R¹ represents aryl, which is substituted with at least one hydroxy group, one carboxy group and/or one carboxy-C₁₋₄-alkyl (HOOC—C₁₋₄-alkyl) group, or R¹ represents C₁₋₆alkyl, aralkyl or C₅r-cycloalkyl, which are substituted with at least one carboxy group, whereby aryl may additionally be substituted with C₁₋₄-alkyl and/or C₁₋₄-alkoxy, and C₁₋₄-alkyl, aralkyl and C₅₋₆-cycloalkyl may additionally be substituted with hydroxy and/or C₁₋₄-alkoxy, and
R² represents hydrogen, C₁₋₄-alkyl, C₅₋₆-cycloalkyl, aryl, aralkyl or R¹, whereby C₁₋₄-alkyl may be substituted with hydroxy, C₁₋₄-alkoxy, carbamoyl and/or cyano, and C₅₋₆cycloalkyl, aryl or aralkyl may be substituted with C₁₋₄-alkyl, hydroxy and/or C₁₋₄-alkoxy,
or
R¹ and R² together the nitrogen to which they are attached complete a piperidino-, pyrrolidinyl- or morpholino ring, which is substituted with at least one carboxy group, and
Z¹ represents C₂₋₆-alkylene or C₅₋₆-cycloalkylene, whereby C₂₋₄-alkylene may be substituted with hydroxy and/or C₁₋₄-alkoxy, and may be interrupted by one or two oxygen atoms, whereby the two oxygens are not linked to each other, and
R³, R⁴ and R⁵ each independently of each other represent hydrogen, C₁₋₄-alkyl, C₅₋₆-cycloalkyl or -Z²-NR⁶R⁷, or R⁴ and R⁵ together with the nitrogen to which they are attached complete a morpholino-, piperidino- or pyrrolidinyl ring, and whereby C₁₋₄-alkyl may be substituted with hydroxy, C₁₋₄-alkoxy, carbamoyl and/or cyano; Z² represents C₂₋₄-alkylene or C₅₋₆-cycloalkylene, whereby C₂₋₄-alkylene may be substituted with hydroxy and/or C₁₋₄-alkoxy, or may be interrupted by one or two oxygen atoms; and R⁶ and R⁷ each independently of each other represent hydrogen, C₁₋₄-alkyl or C₅₋₆cycloalkyl,
or
Z¹, R³ and R⁴ together with the nitrogens to which they are attached complete a piperazinyl ring,
with the proviso that when R¹ represents 4-carboxyphenyl and R² represents hydrogen, Z¹ is not ethylene, R⁴ and R⁵ are not methyl and R³ is not hydrogen, or Z¹ is not trimethylene, R⁴ and R⁵ do not together with the nitrogen to which they are attached complete a morpholino ring and R³ is not hydrogen.

2. The 4,4′-bis(triazinylamino)stilbene-2,2′-disulfonic acid derivative of claim 1 or salts thereof, in which
R¹ represents phenyl which is substituted with at least one hydroxy group, one carboxy group and/or one carboxy-C₁₋₄-alkyl (HOOC—C₁₋₄-alkyl) group, or R¹ represents C₁₋₆alkyl which is substituted with at least one carboxy group, whereby phenyl may additionally be substituted with C₁₋₄-alkyl and/or C₁₋₄-alkoxy, and C₁₋₆alkyl may additionally be substituted with hydroxy and/or C₁₋₄-alkoxy, and
R² represents hydrogen, C₁₋₄-alkyl, or R¹, whereby C₁₋₄-alkyl may be substituted with hydroxy, C₁₋₄-alkoxy, carbamoyl and/or cyano, and
Z¹ represents C₂₋₄-alkylene, which may be substituted with hydroxy and/or
C₁₋₄-alkoxy, or may be interrupted by one or two oxygen atoms, and
R³ represents hydrogen or C₁₋₄-alkyl, whereby C₁₋₄-alkyl may be substituted with hydroxy, C₁₋₄-alkoxy, carbamoyl and/or cyano, and
R⁴ and R⁵ each independently of each other represent hydrogen, C₁₋₄-alkyl or -Z²-NR⁶R⁷, or together with the nitrogen to which they are attached complete a morpholino-, piperidino- or pyrrolidinyl ring, and whereby C₁₋₄-alkyl may be substituted with hydroxy and/or C₁₋₄-alkoxy; Z² represents C₂₋₄-alkylene, which may be substituted with hydroxy and/or C₁₋₄-alkoxy, and may be interrupted by one or two oxygen atoms, whereby the two oxygens are not linked to each other; and R⁶ and R⁷ each independently of each other represent hydrogen or C₁₋₄-alkyl;
or Z¹, R³ and R⁴ together with the nitrogens to which they are attached complete a piperazinyl ring.

3. The 4,4′-bis(triazinylamino)stilbene-2,2′-disulfonic acid derivative of claim 2, or salts thereof, in which
R¹ represents phenyl which is substituted with at least one hydroxy group and/or one carboxy group, or R¹ represents C₁₋₆alkyl, which are substituted with at least one carboxy group, whereby phenyl may additionally be substituted with C₁₋₄-alkyl, C₁₋₄-alkoxy and/or C₁₋₄-alkylsulfonyl, and C₁₋₆alkyl may additionally be substituted with hydroxy and/or C₁₋₄-alkoxy, and
R² represents hydrogen, C₁₋₄-alkyl or R¹; Z¹ represents C₂₋₄-alkylene, whereby C₂₋₄-alkylene may be substituted with hydroxy and/or C₁₋₄-alkoxy, and
R³ represents hydrogen or C₁₋₄-alkyl, whereby C₁₋₄-alkyl may be substituted with hydroxy and/or C₁₋₄-alkoxy, and
R⁴ and R⁵ each independently of each other represent hydrogen, C₁₋₄-alkyl or -Z²-NR⁶R⁷, whereby C₁₋₄-alkyl may be substituted with hydroxy and/or C₁₋₄-alkoxy; or
R⁴ and R⁵ together with the nitrogen to which they are attached complete a morpholino ring; Z² represents C₂₋₄-alkylene, which may be substituted with hydroxy and/or C₁₋₄-alkoxy; and R⁶ and R⁷ each independently of each other represent hydrogen or C₁₋₄-alkyl,
or Z¹, R³ and R⁴ together with the nitrogens to which they are attached complete a piperazinyl ring.

4. 4,4′-bis(triazinylamino)stilbene-2,2′-disulfonic acid derivative of claim 3, or salts thereof, in which
R¹ represents phenyl or C₁₋₆-alkyl, which are substituted with one hydroxy or carboxy group, and
R² represents hydrogen, methyl or R¹; Z¹ represents C₂₋₃-alkylene, and
R³ represents hydrogen, and
R⁴ and R⁵ each independently of each other represent hydrogen or C₁₋₄-alkyl, whereby C₁₋₄-alkyl may be substituted with hydroxy; or R⁴ and R⁵ together with the nitrogen to which they are attached complete a morpholino ring,
or Z¹, R³ and R⁴ together with the nitrogens to which they are attached complete a piperazinyl ring.

5. A composition comprising 4,4′-bis(triazinylamino)stilbene-2,2′-disulfonic acid derivatives of the formulae 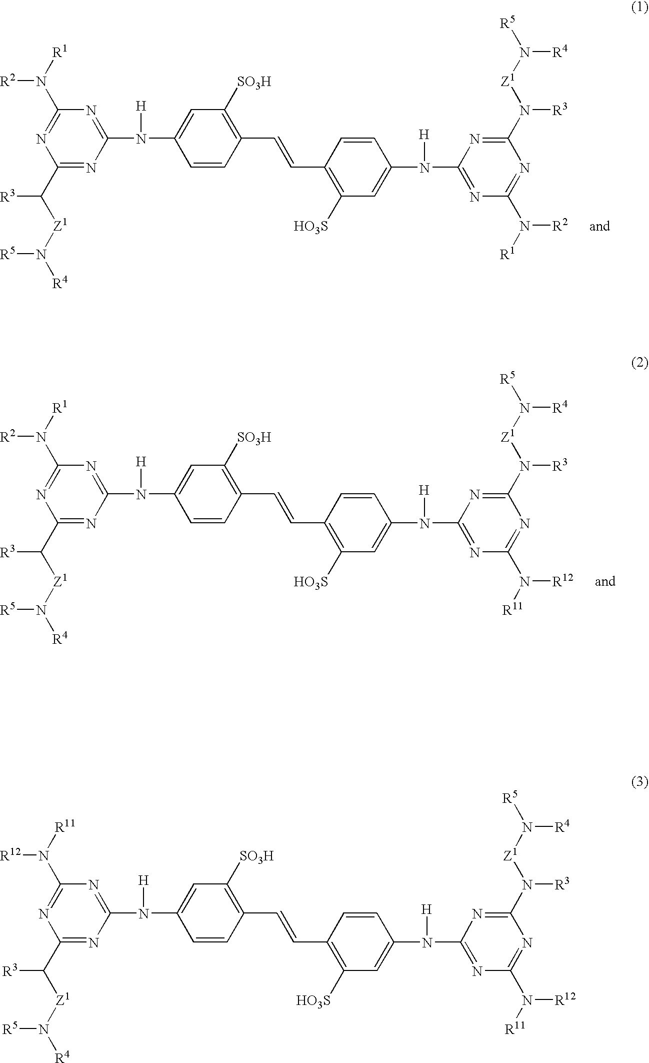
or salts thereof, in which R¹, R², Z¹, R³, R⁴ and R⁵ are as defined in claim 1 and
R¹¹ represents aryl, C₁₋₆alkyl, aralkyl or C₅₋₆-cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆cycloalkyl, aryl, aralkyl or R¹¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy.

6. The composition of claim 5, in which
R¹ represents phenyl which is substituted with at least one hydroxy group, one carboxy group and/or one carboxy-C₁₋₄-alkyl (HOOC—C₁₋₄-alkyl) group, or R¹ represents C₁₋₆alkyl which is substituted with at least one carboxy group, whereby phenyl may additionally be substituted with C₁₋₄-alkyl and/or C₁₋₄-alkoxy, and C₁₋₆-alkyl may additionally be substituted with hydroxy and/or C₁₋₄-alkoxy, and
R² represents hydrogen, C₁₋₄-alkyl, or R¹, whereby C₁₋₄-alkyl may be substituted with hydroxy, C₁₋₄-alkoxy, carbamoyl and/or cyano, and
Z¹ represents C₂₋₆-alkylene, which may be substituted with hydroxy and/or C₁₋₄-alkoxy, or may be interrupted by one or two oxygen atoms, and
R³ represents hydrogen or C₁₋₄-alkyl, whereby C₁₋₄₄-alkyl may be substituted with hydroxy, C₁₋₄-alkoxy, carbamoyl and/or cyano, and
R⁴ and R⁵ each independently of each other represent hydrogen, C₁₋₄-alkyl or -Z²-NR⁶R⁷, or together with the nitrogen to which they are attached complete a morpholino-, Piperidino- or Pyrrolidinylring, and whereby C₁₋₄-alkyl may be substituted with hydroxy and/or C₁₋₄-alkoxy; Z² represents C₂₋₆-alkylene, which may be substituted with hydroxy and/or C₁₋₄-alkoxy, and may be interrupted by one or two oxygen atoms, whereby the two oxygens are not linked to each other; and R⁶ and R⁷ each independently of each other represent hydrogen or C₁₋₄-alkyl; or Z¹, R³ and R⁴ together with the nitrogens to which they are attached complete a piperazinyl ring,
and
R¹¹ represents phenyl or C₁₋₆alkyl, whereby phenyl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆alkyl may be substituted with a hydroxy, a carboxy group, C₁₋₄-alkoxy, carbamoyl and/or cyano; and
R¹² represents hydrogen, C₁₋₄-alkyl or R¹¹, whereby C₁₋₄-alkyl may be substituted with hydroxy, C₁₋₄-alkoxy, carbamoyl and/or cyano.

7. The composition of claim 6, in which
R¹ represents phenyl which is substituted with at least one hydroxy group and/or one carboxy group, or R¹ represents C₁₋₆-alkyl, which are substituted with at least one carboxy group, whereby Phenyl may additionally be substituted with C₁₋₄-alkyl, C₁₋₄-alkoxy and/or C₁₋₄-alkylsulfonyl, and C₁₋₄-alkyl may additionally be substituted with hydroxy and/or C₁₋₄-alkoxy, and
R² represents hydrogen. C₁₋₄-alkyl or R¹; Z¹ represents C₂₋₄-alkylene, whereby C₂₋₄-alkylene may be substituted with hydroxy and/or C₁₋₄-alkoxy, and
R³ represents hydrogen or C₁₋₄-alkyl, whereby C₁₋₄-alkyl may be substituted with hydroxy and/or C₁₋₄-alkoxy, and
R⁴ and R⁵ each independently of each other represent hydrogen, C₁₋₄-alkyl or -Z²-NR⁶R′, whereby C₁₋₄-alkyl may be substituted with hydroxy and/or C₁₋₄-alkoxy; or R⁴ and R⁵ together with the nitrogen to which they are attached complete a morpholino ring; Z² represents C₂₋₄-alkylene, which may be substituted with hydroxy and/or C₁₋₄-alkoxy; and R⁶ and R⁷ each independently of each other represent hydrogen or C₁₋₄-alkyl,
or Z¹, R³ and R⁴ together with the nitrogens to which they are attached complete a piperazinyl ring;
R¹¹ represents phenyl, whereby phenyl may be substituted with a hydroxy group, a carboxy group, a carboxy-C₁₋₄-alkyl (HOOC—C₁₋₄-alkyl) group, C₁₋₄-alkyl and/or C₁₋₄-alkoxy; and R¹² represents hydrogen or C₁₋₄-alkyl.

8. The composition of claim 7, in which
R¹ represents phenyl which is substituted with at least one hydroxy group and/or one carboxy group, or R¹ represents C₁₋₆alkyl, which are substituted with at least one carboxy group, whereby phenyl may additionally be substituted with C₁₋₄-alkyl, C₁₋₄-alkoxy and/or C₁₋₄-alkylsulfonyl, and C₁₋₄-alkyl may additionally be substituted with hydroxy and/or C₁₋₄-alkoxy, and
R² represents hydrogen, C₁₋₄-alkyl or R¹; Z¹ represents C₂₋₄-alkylene, whereby C₂₋₄-alkylene may be substituted with hydroxy and/or C₁₋₄-alkoxy, and
R³ represents hydrogen or C₁₋₄-alkyl, whereby C₁₋₄-alkyl may be substituted with hydroxy and/or C₁₋₄-alkoxy, and
R⁴ and R⁵ each independently of each other represent hydrogen, C₁₋₄-alkyl or -Z²-NR⁶R⁷, whereby C₁₋₄-alkyl may be substituted with hydroxy and/or C₁₋₄-alkoxy; or R⁴ and R⁵ together with the nitrogen to which they are attached complete a morpholino ring; Z² represents C₂₋₄-alkylene, which may be substituted with hydroxy and/or C₁₋₄-alkoxy; and R⁶ and R⁷ each independently of each other represent hydrogen or C₁₋₄-alkyl,
or Z¹, R³ and R⁴ together with the nitrogens to which they are attached complete a Piperazinyl ring;
R¹¹ represents phenyl, which is unsubstituted or substituted with one carboxy group; and R¹² represents hydrogen.

9. A 4,4′-bis(triazinylamino)stilbene-2,2′-disulfonic acid derivatives of the formula 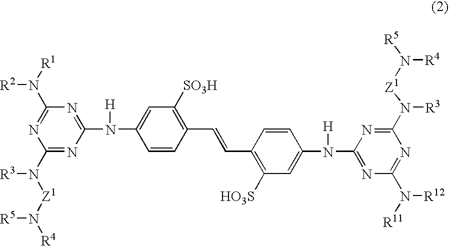
or salts thereof, in which R¹, R², Z¹, R³, R⁴ and R⁵ are as defined in claim 1, and
R¹¹ represents aryl, C₁₋₆alkyl, aralkyl or CF₆-cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₄-alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆-cycloalkyl, aryl, aralkyl or R¹¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy.

10. A process for the preparation of the 4,4′-bis(triazinylamino)stilbene-2,2′-disulfonic acid derivatives of the formula 1 according to claim 1, comprising the steps of
i) reacting a 4,4′-bis[(4,6-dihalotriazinyl)amino]stilbene-2,2′-disulfonic acid derivative of the formula 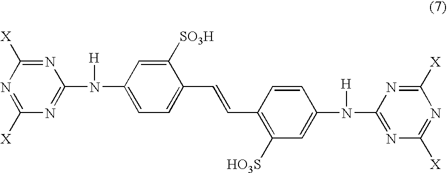
or salts thereof, in which X represents bromine, chlorine, fluorine or iodine,
with an amine of the formula HNR¹R² (8), in which R¹ and R² have the meaning as indicated in claim 1, to yield a 4,4′-bis(triazinylamino)stilbene-2,2′-disulfonic acid derivative of the formula 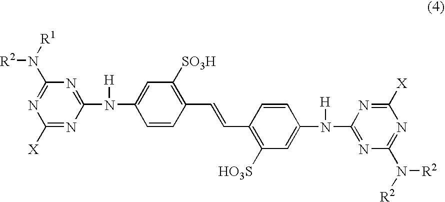
or salts thereof, in which R¹ and R² have the meaning as indicated in claim 1
ii) reacting the 4,4′-bis(triazinylamino)stilbene-2,2′-disulfonic acid derivative of the formula 4 or salts thereof obtained in step i) with an amine of the formula HNR³(Z¹-NR⁴R⁵) (9), in which R³, Z¹, R⁴ and R⁵ have the meaning as indicated above, to yield the 4,4′-bis(triazinylamino)stilbene-2,2′-disulfonic acid derivative of the formula 1.

11. A process for the preparation of the composition comprising 4,4′-bis(triazinylamino)stilbene-2,2′-disulfonic acid derivatives of the formulae 1, 2 and 3 or salts thereof according to claim 5 comprising the steps of
i) reacting a 4,4′-bis[(4,6-dihalotriazinylamino)]stilbene-2,2′-disulfonic acid derivate of the formula 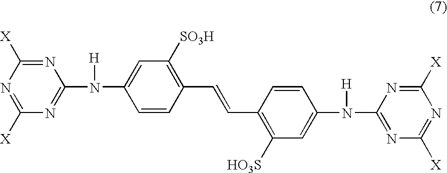
or salts thereof, in which X represents bromine, chlorine, fluorine or iodine,
with a mixture of amines of the formulae HNR¹R² (8), and HNR¹¹R¹² (10), in which
R¹ represents phenyl which is substituted with at least one hydroxy group, one carboxy group and/or one carboxy-C₁₋₄-alkyl (HOOC—C₁₋₄-alkyl) group, or R¹ represents C₁₋₆-alkyl which is substituted with at least one carboxy group, whereby phenyl may additionally be substituted with C₁₋₄-alkyl and/or C₁₋₄-alkoxy, and C₁₋₆alkyl may additionally be substituted with hydroxy and/or C₁₋₄-alkoxy, and
R² represents hydrogen, C₁₋₄-alkyl, or R¹, whereby C₁₋₄-alkyl may be substituted with hydroxy, C₁₋₄-alkoxy, carbamoyl and/or cyano or R¹ and R² together the nitrogen to which they are attached complete a piperidino-, pyrrolidinyl- or morpholino ring, which is substituted with at least one carboxy group, and
R¹¹ represents aryl, C₁₋₆alkyl, aralkyl or C₅₋₆-cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆cycloalkyl, aryl, aralkyl or R¹¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy,
to yield a composition comprising 4,4′-bis(triazinylamino)stilbene-2,2′-disulfonic acid derivatives of the formulae 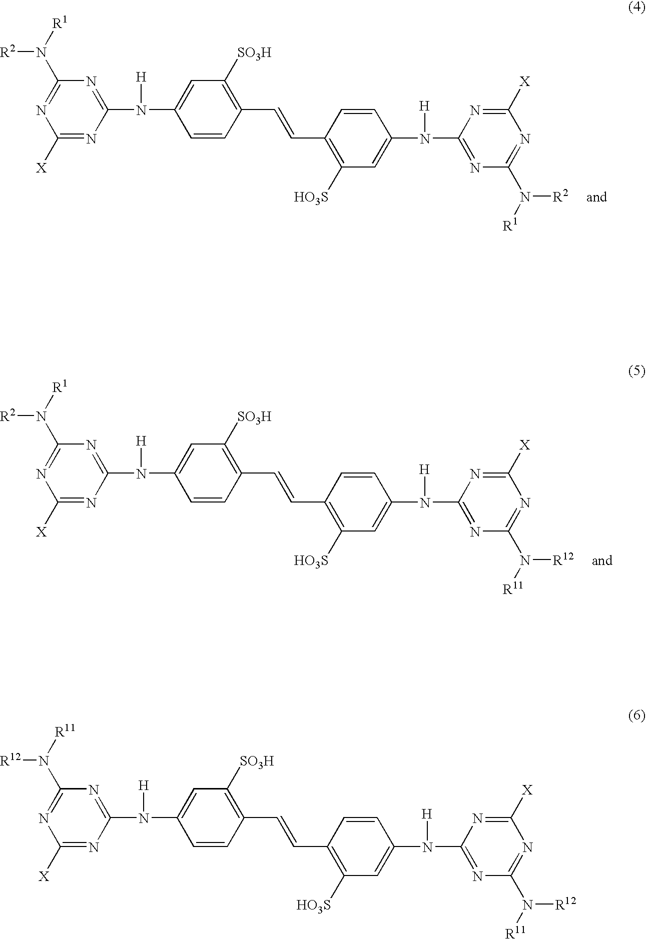
or salts thereof, in which X represents bromine, chlorine, fluorine or iodine,
ii) reacting the composition comprising 4,4′-bis(triazinylamino)stilbene-2,2′-disulfonic acid derivatives of the formulae 4, 5 and 6 obtained in step i) with an amine of the formula HNR³(Z¹-NR⁴R⁵) (9), in which R³, Z¹, R⁴ and R⁵ are as defined as in claim 5, to yield the composition of 4,4′-bis(triazinylamino)stilbene-2,2′-disulfonic acid derivatives of the formulae 1, 2 and 3.

12. Intermediate 4,4′-bis(triazinylamino)stilbene-2,2′-disulfonic acid derivatives of the formulae 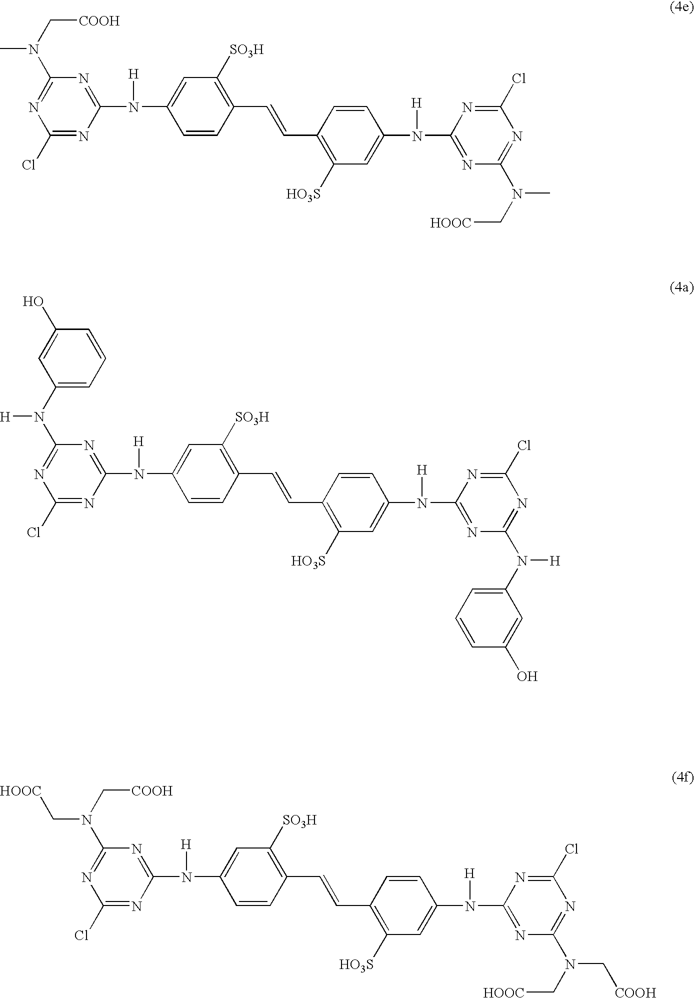
or salts thereof.

13. A composition of intermediate 4,4′-bis(triazinylamino)stilbene-2,2′-disulfonic acid derivatives of the formulae 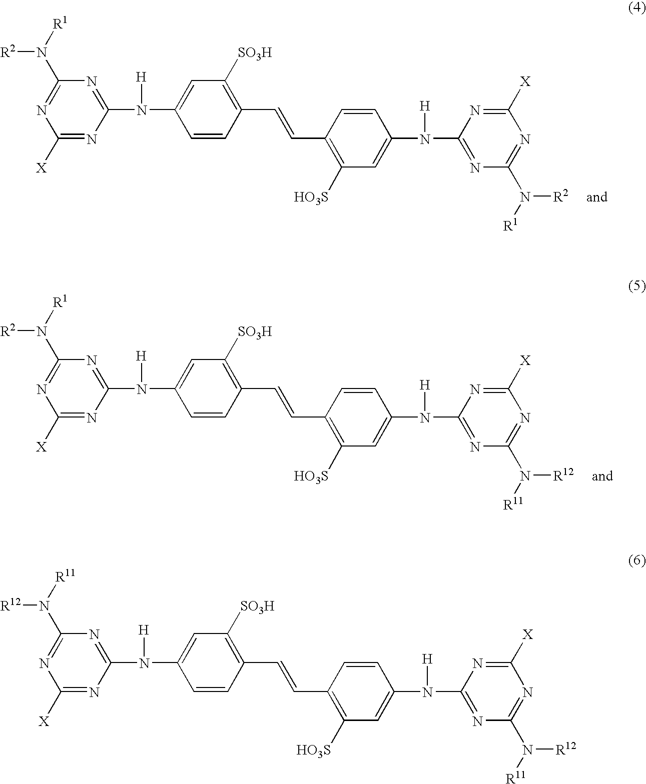
or salts thereof, in which R¹ and R² have the meaning as indicated in claim 1 and
R¹¹ represents aryl, C₁₋₆alkyl, aralkyl or C₅₋₆-cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆-cycloalkyl, aryl, aralkyl or R¹¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy,
and X represents bromine, chlorine, fluorine or iodine.

14. An intermediate 4,4′-bis(triazinylamino)stilbene-2,2′-disulfonic acid derivative of the formula 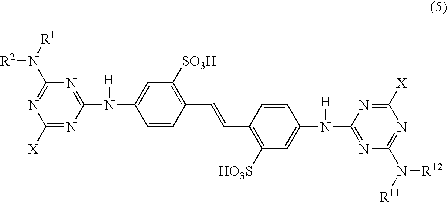
or salts thereof, in which R¹ and R² have the meaning as indicated in claim 1,
R¹¹ represents aryl, C₁₋₆alkyl, aralkyl or C₅₋₆-cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆-alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆-cycloalkyl, aryl, aralkyl or R¹¹, whereby C₁₋₄-alkyl may be substituted with hydroxy. C₁₋₄-alkoxy, carbamoyl and/or cyano, and C₅₋₆-cycloalkyl, aryl or aralkyl may be substituted with C₁₋₄-alkyl, hydroxy and/or and X represents bromine, chlorine, fluorine or iodine.

15. A method of brightening paper by treating paper with 4,4′-bis(triazinylamino)stilbene-2,2′-disulfonic acid derivative of the formulae 1 or 2 or salts thereof according to claims 1, or by treating paper with the composition comprising 4,4′-bis(triazinylamino)stilbene-2,2′-disulfonic acid derivative of the formula 1, 2 and 3 or salts thereof as defined below 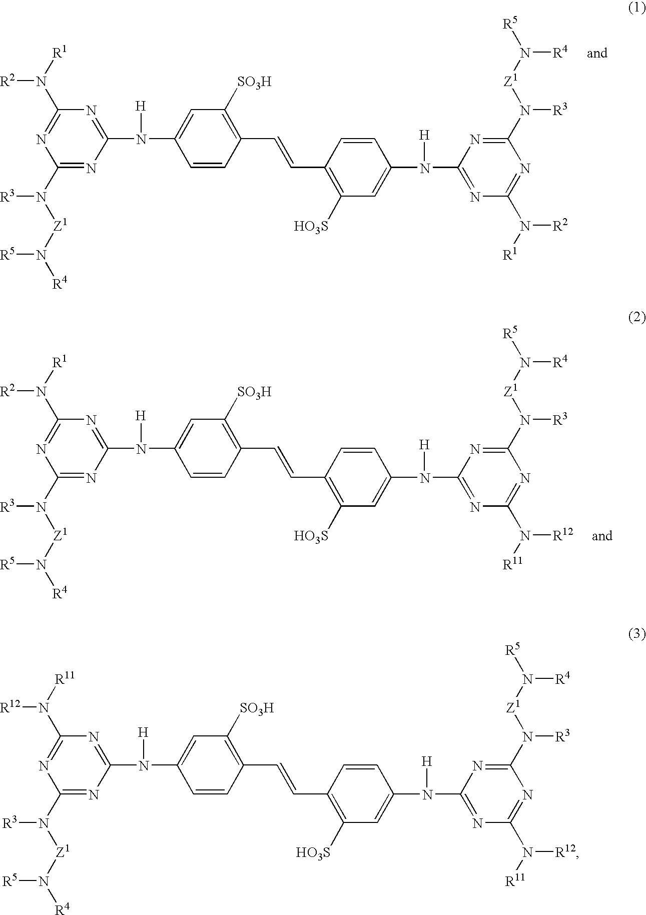
in which R¹, R. Z. R³, R⁴ and R⁵ are as defined in claim 1 and
R¹¹ represents aryl, C₁₋₆-alkyl, aralkyl or C₅₋₆cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆-alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆-cycloalkyl, aryl, aralkyl or R¹¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy.

16. Paper treated with the 4,4′-bis(triazinylamino)stilbene-2,2′-disulfonic acid derivative of the formulae 1 or 2 or salts thereof according to claims 1, or with the composition of 4,4′-bis(triazinylamino)stilbene-2,2′-disulfonic acid derivatives of the formulae 1, 2 and 3 or salts thereof as defined below 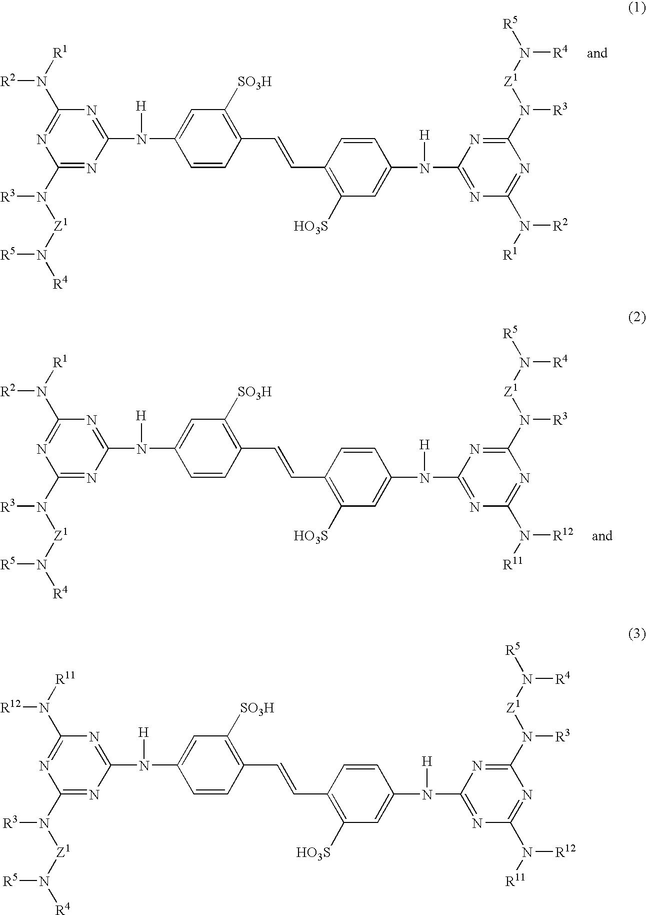
or salts thereof, in which R¹, R², Z¹. R³. R⁴ and R⁵ are as defined in claim 1 and
R¹¹ represents aryl, C₁₋₆alkyl, aralkyl or C₅₋₆cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆-alkyl, aralkyl and C₅₋₆-cycloalkyl may be substituted with a hydroxy, a carboxy group. C₁₋₄-alkoxy, carbamoyl and/or cyano, and
R¹² represents hydrogen. C₁₋₄-alkyl, C₅₋₆ cycloalkyl, aryl, aralkyl or R¹¹, whereby C₁₋₄-alkyl may be substituted with hydroxy, C₁₋₄-alkoxy, carbamoyl and/or cyano, and C₅₋₆cycloalkyl, aryl or aralkyl may be substituted with C₁₋₄-alkyl, hydroxy and/or C₁₋₄-alkoxy.

17. An aqueous formulation comprising the 4,4′-bis(triazinylamino)stilbene-2,2′-disulfonic acid derivative of the formulae 1 or 2 or salts thereof according to claims 1, or the composition of 4,4′-bis(triazinylamino)stilbene-2,2′-disulfonic acid derivatives of the formulae 1, 2 and 3 or salts thereof as defined below 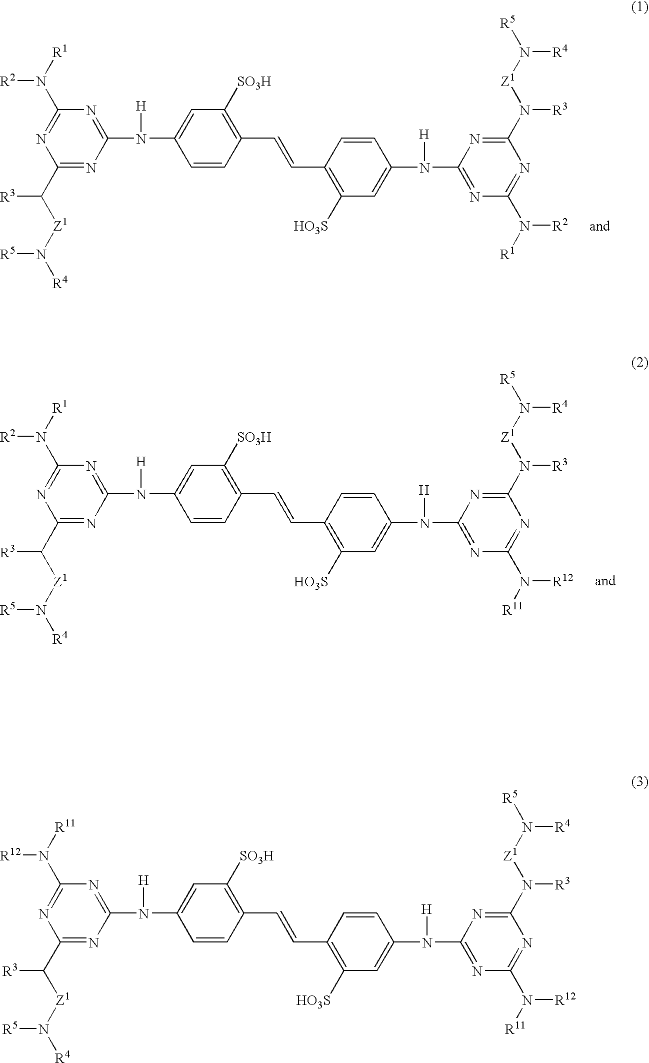
or salts thereof, in which R¹, R², Z¹. R³, R⁴ and R⁵ are as defined in claim 1 and
R¹¹represents aryl, C₁₋₄-alkyl, aralkyl or C₅₋₆-cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₄-alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄ alkyl, C₅₋₆cycloalkyl, aryl, aralkyl or R¹¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy.

18. A method of brightening paper by treating paper with 4,4′-bis(triazinylamino)stilbene-2,2′-disulfonic acid derivative of the formulae 1 or 2 or salts thereof according to claims 9, or by treating paper with the composition comprising 4,4′-bis(triazinylamino)stilbene-2,2′-disulfonic acid derivative of the formula 1, 2 and 3 or salts thereof as defined below 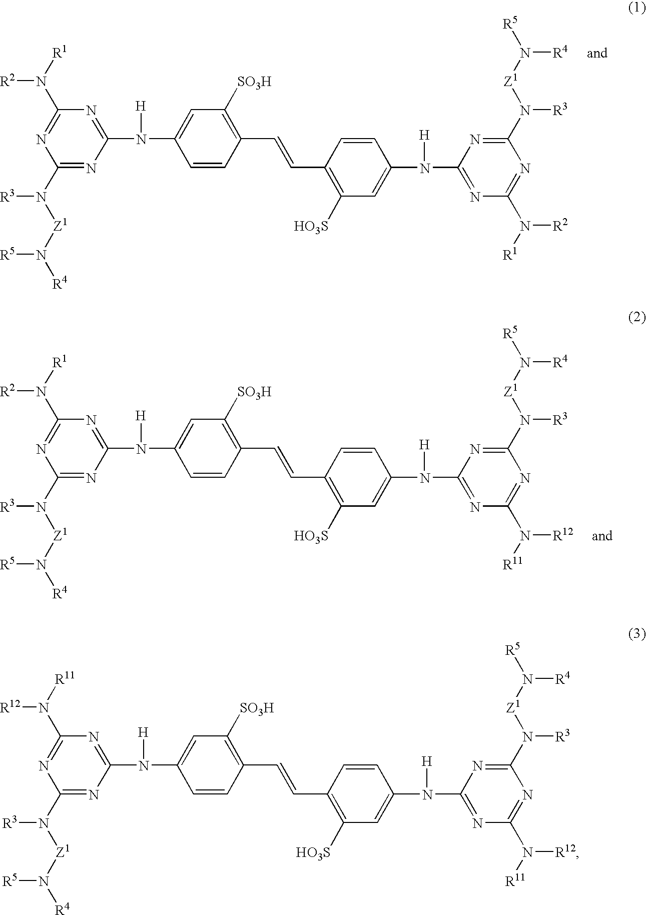
in which R¹, R², Z¹, R³, R⁴ and R⁵ are as defined in claim 1 and
R¹¹represents aryl, C₁₋₆-alkyl, aralkyl or C₅₋₆-cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆-alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆-cycloalkyl, aryl, aralkyl or R¹¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy.

19. Paper treated with the 4,4′-bis(triazinylamino)stilbene-2,2′-disulfonic acid derivative of the formulae 1 or 2 or salts thereof according to claims 9, or with the composition of 4,4′-bis(triazinylamino)stilbene-2,2′-disulfonic acid derivatives of the formulae 1, 2 and 3 or salts thereof as defined below 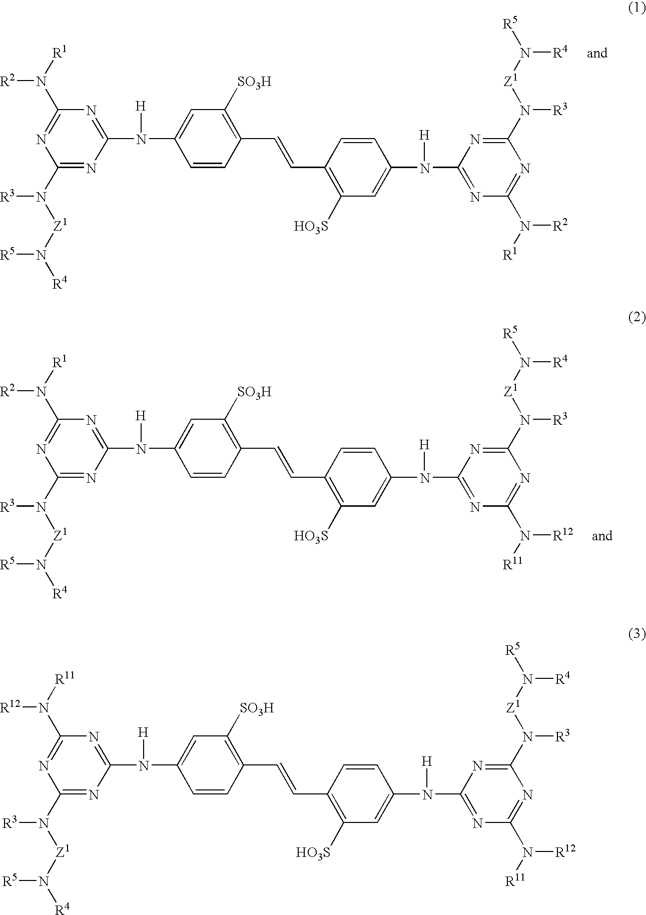
or salts thereof, in which R¹, R², Z¹, R³, R⁴ and R⁵ are as defined in claim 1 and
R¹¹ represents aryl, C₁₋₆alkyl, aralkyl or C₅₋₆-cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₄-alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆-cycloalkyl, aryl, aralkyl or R¹¹, whereby C₁₋₄-alkyl may be substituted with hydroxy, C₁₋₄-alkoxy, carbamoyl and/or cyano, and C₅₋₆-cycloalkyl, aryl or aralkyl may be substituted with C₁₋₄-alkyl, hydroxy and/or C₁₋₄-alkoxy.

20. An aqueous formulation comprising the 4,4′-bis(triazinylamino)stilbene-2,2′-disulfonic acid derivative of the formulae 1 or 2 or salts thereof according to claims 9, or the composition of 4,4′-bis(triazinylamino)stilbene-2,2′-disulfonic acid derivatives of the formulae 1, 2 and 3 or salts thereof as defined below 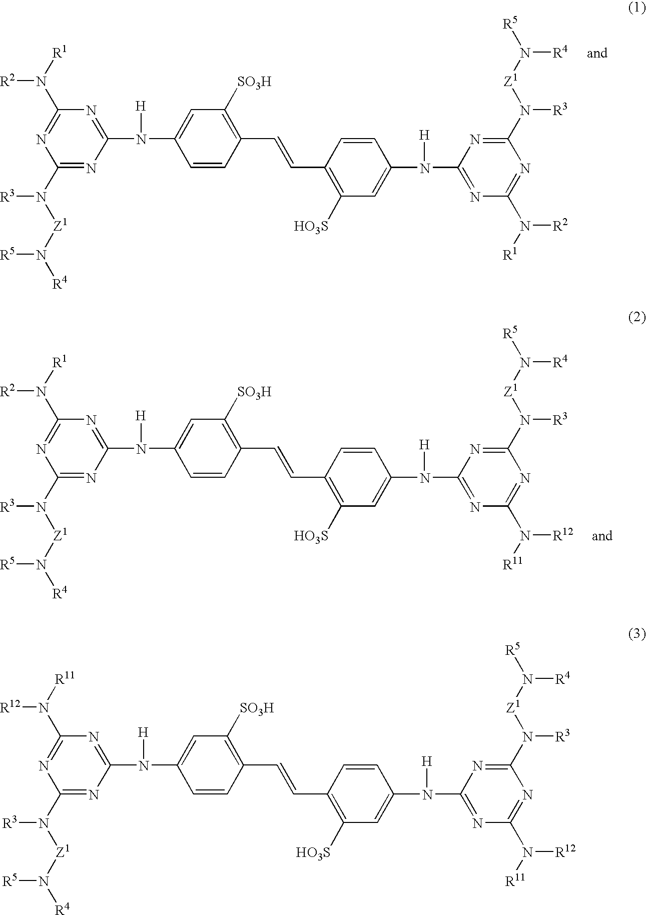
or salts thereof, in which R¹, R², Z¹, R³, R⁴ and R⁵ are as defined in claim 1 and
R¹¹ represents aryl, C₁₋₆alkyl, aralkyl or C₅₋₆cycloalkyl, whereby aryl may be substituted with a hydroxy group, a carboxy group, a carboxy-C₁₋₄-alkyl (HOOC—C₁₋₄-alkyl) group, C₁₋₄-alkyl and/or C₁₋₄-alkoxy, and C₁₋₆-alkyl, aralkyl and C₅₋₆-cycloalkyl may be substituted with a hydroxy, a carboxy group, C₁₋₄-alkoxy, carbamoyl and/or cyano, and
R¹² represents hydrogen, C₁₋₄-alkyl, C₅₋₆cycloalkyl, aryl, aralkyl or R¹¹, whereby C₁₋₄-alkyl may be substituted with hydroxy, C₁₋₄-alkoxy, carbamoyl and/or cyano, and C₅₋₆cycloalkyl, aryl or aralkyl may be substituted with C₁₋₄-alkyl, hydroxy and/or C₁₋₄-alkoxy.
